(19) United States
(12) Patent Application Publication
(10) Pub. No.: US 2023/0360737 A1
(43) Pub. Date: Nov. 9, 2023

(54) CHEMICAL COMPOUND RECOMMENDATION PROCESS

(71) Applicant: TOYOTA RESEARCH INSTITUTE, INC., Los Altos, CA (US)

(72) Inventors: Steven TORRISI, Berkeley, CA (US); Joseph H. MONTOYA, Berkeley, CA (US)

(73) Assignees: TOYOTA RESEARCH INSTITUTE, INC., Los Altos, CA (US); TOYOTA JIDOSHA KABUSHIKI KAISHA, Aichi-Ken (JP)

(21) Appl. No.: 17/739,932

(22) Filed: May 9, 2022

(51) Int. Cl. G16C 20/10

(52) U.S. Cl. CPC G16C 20/10

(57) ABSTRACT

A method for setting parameters for a chemical reaction includes receiving a target material and a set of available substrate structures to host the target material. The method also includes filtering stored thermodynamic parameters associated with chemical compounds based on receiving the target material and the set of available substrate structures. The method further includes generating a recommendation indicating a precursor electrolyte, a substrate structure from the set of available substrate structures, and electrochemical conditions for obtaining the target material, the recommendation may be generated based on filtering the stored thermodynamic parameters.

### BACKGROUND

### Field

Certain aspects of the present disclosure generally relate to chemical reaction workflows, more specifically, certain aspects of the present disclosure relate to systems and methods for recommending a chemical compound for a workflow.

### Background

In some cases, an electrochemical reactor may be used for chemical synthesis. In such cases, a solution may be exposed to a substrate in contact with an electrode. The electrode may be set to a bias voltage. Additionally, the solution may be an aqueous medium with a dissolved ionic species. A state of both the electrochemical reactor and a resultant equilibrium product may be determined by an acidity of the solution, a concentration and type of ions in an electrolyte, a voltage of the electrode, and a compound of the substrate. It may be desirable to improve the chemical synthesis process by determining a set of relevant reaction precursors and recommending a strategy for the synthesis, or extraction, of a target chemical compound from the substrate or solution based on a database of chemical compounds and associated thermochemical parameters.

### SUMMARY

In one aspect of the present disclosure, a method for setting parameters for a chemical reaction includes receiving a target material and a set of available substrate structures to host the target material. The method further includes filtering stored thermodynamic parameters associated with chemical compounds based on receiving the target material and the set of available substrate structures. The method still further includes generating a recommendation indicating a precursor electrolyte, a substrate structure from the set of available substrate structures, and electrochemical conditions for obtaining the target material, the recommendation being generated based on filtering the stored thermodynamic parameters.

Another aspect of the present disclosure is directed to an apparatus including means for receiving a target material and a set of available substrate structures to host the target material. The apparatus further includes means for filtering stored thermodynamic parameters associated with chemical compounds based on receiving the target material and the set of available substrate structures. The apparatus still further includes means for generating a recommendation indicating a precursor electrolyte, a substrate structure from the set of available substrate structures, and electrochemical conditions for obtaining the target material, the recommendation being generated based on filtering the stored thermodynamic parameters.

In another aspect of the present disclosure, a non-transitory computer-readable medium with non-transitory program code recorded thereon is disclosed. The program code is executed by a processor and includes program code to receive a target material and a set of available substrate structures to host the target material. The program code further includes program code to filter stored thermodynamic parameters associated with chemical compounds based on receiving the target material and the set of available substrate structures. The program code still further includes program code to generate a recommendation indicating a precursor electrolyte, a substrate structure from the set of available substrate structures, and electrochemical conditions for obtaining the target material, the recommendation being generated based on filtering the stored thermodynamic parameters.

Another aspect of the present disclosure is directed to an apparatus having a processor, and a memory coupled with the processor and storing instructions operable, when executed by the processor, to cause the apparatus to receive a target material and a set of available substrate structures to host the target material. Execution of the instructions also cause the apparatus to filter stored thermodynamic parameters associated with chemical compounds based on receiving the target material and the set of available substrate structures. Execution of the instructions further cause the apparatus to generate a recommendation indicating a precursor electrolyte, a substrate structure from the set of available substrate structures, and electrochemical conditions for obtaining the target material, the recommendation being generated based on filtering the stored thermodynamic parameters.

This has outlined, rather broadly, the features and technical advantages of the present disclosure in order that the detailed description that follows may be better understood. Additional features and advantages of the present disclosure will be described below. It should be appreciated by those skilled in the art that this present disclosure may be readily utilized as a basis for modifying or designing other structures for carrying out the same purposes of the present disclosure. It should also be realized by those skilled in the art that such equivalent constructions do not depart from the teachings of the present disclosure as set forth in the appended claims. The novel features, which are believed to be characteristic of the present disclosure, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

The features, nature, and advantages of the present disclosure will become more apparent from the detailed description set forth below when taken in conjunction with the drawings in which like reference characters identify correspondingly throughout.

FIG. 1 is a block diagram illustrating an example of an electrochemical reactor, in accordance with various aspects of the present disclosure.

FIG. 2 is a block diagram illustrating an example of a Pourbaix diagram, in accordance with various aspects of the present disclosure.

FIG. 3 is a flow diagram illustrating an example of a process 300 for recommending a chemical compound, in accordance with various aspects of the present disclosure.

FIG. 4 is a block diagram illustrating an example of a physical reactor system, in accordance with aspects of the present disclosure.

FIG. 5 is a diagram illustrating an example of a hardware implementation for component of a physical reactor system, according to aspects of the present disclosure.

FIG. 6 illustrates a flow diagram for a method according to aspects of the present disclosure.

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. It will be apparent to those skilled in the art, however, that these concepts may be practiced without these specific details. In some instances, well-known structures and components are shown in block diagram form in order to avoid obscuring such concepts.

As discussed, in some cases, an electrochemical reactor may be used for chemical synthesis. In such cases, a solution may be exposed to a substrate in contact with an electrode. The electrode may be set to a bias voltage. Additionally, the solution may be an aqueous medium with a dissolved ionic species. A state of both the electrochemical reactor and a resultant equilibrium product may be determined by an acidity of the solution, a concentration and type of ions in an electrolyte, a voltage of the electrode, and a compound of the substrate. It may be desirable to improve the chemical synthesis process by determining a set of relevant reaction precursors and recommending a strategy for the synthesis, or extraction, of a target chemical compound from the substrate or solution based on a database of chemical compounds and associated thermochemical parameters.

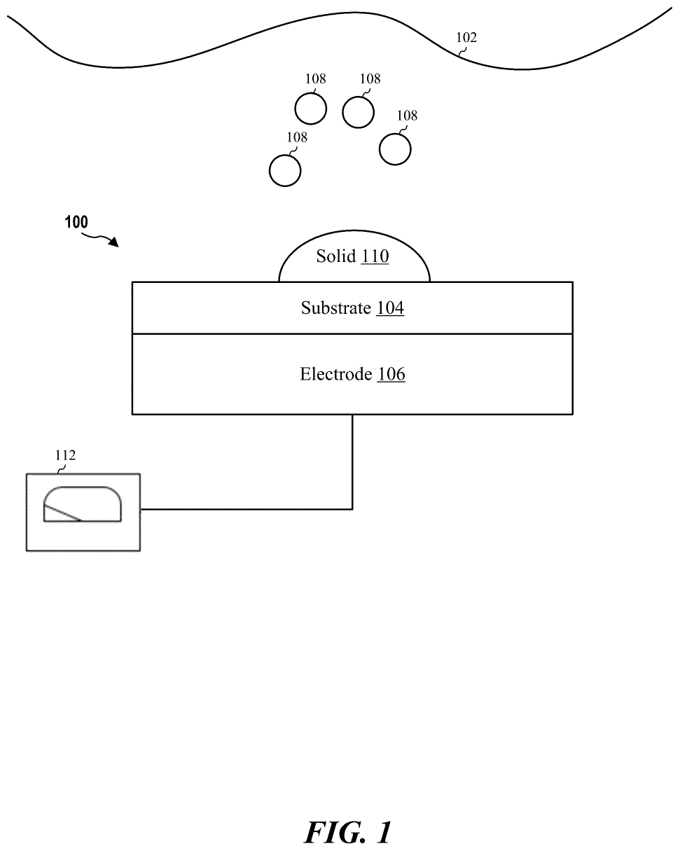

FIG. 1 is a block diagram illustrating an example of an electrochemical reactor 100, in accordance with various aspects of the present disclosure. In the example of FIG. 1, the electrochemical reactor 100 may include a substrate 104 in contact with an electrode 106. The substrate may be exposed to an aqueous medium 102 with dissolved ionic species 108 (e.g., aqueous ions). The aqueous medium may be referred to as an aqueous ion solution. In the example of FIG. 1, four dissolved ionic species 108 are shown for illustrative purposes, aspects of the present disclosure are not limited to only four dissolved ionic species 108. The electrode 106 may be set to a bias voltage. The voltage may be provided via a power supply 112. A state of the reactor 100, and a resultant equilibrium product, such as a solid 110, may be determined by the acidity of the aqueous medium 102 (e.g., a solution), a concentration and a type of ions 108, the voltage of the electrode 106, and a compound of the substrate 104.

Aspects of the present disclosure are directed to a process for determining a set of relevant reaction precursors and recommending a strategy for the synthesis or extraction of a target chemical compound, such as the solid 110, from the substrate 104 or solution 102. The set of relevant reaction precursors and the recommended strategy may be based on a database of chemical compounds and associated thermochemical parameters. In some examples, the process may apply a recommended voltage and autonomously adjust an amount of solution recommended for synthesis or extraction of the target chemical compound.

In some examples, a Pourbaix diagram may be used to visually organize thermodynamic equilibria associated with a system of interacting chemical elements. The Pourbaix diagram may be partitioned into regions that describe thermodynamically stable species and encode information associated with condition thresholds where the most stable species change. Additionally, the Pourbaix diagram may show equilibrium conditions for a set of electrolyte and substrate phases, and how these phases change in response to changing operating conditions. In some examples, the Pourbaix diagram may be compiled by comparing entries of experimentally or theoretically obtained thermodynamic parameters.

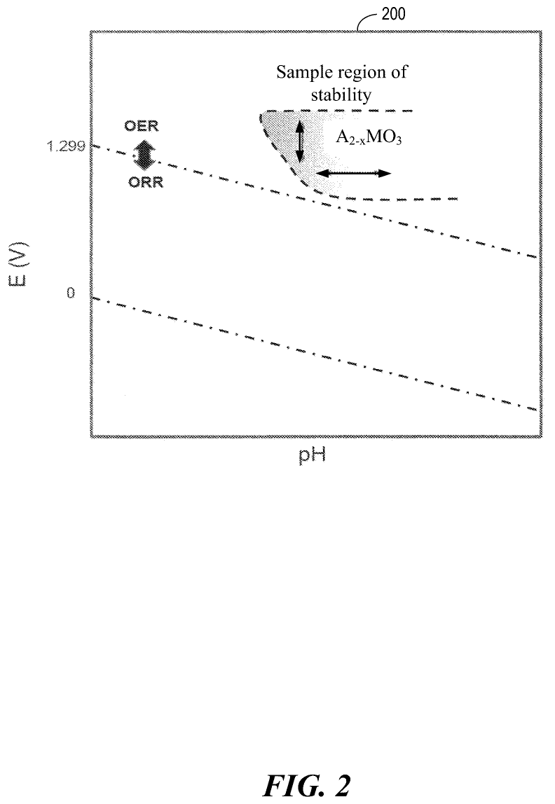

FIG. 2 is a block diagram illustrating an example of a Pourbaix diagram 200, in accordance with various aspects of the present disclosure. In the example of FIG. 2, the Pourbaix diagram 200 is used in a process for maintaining electrochemical stability of an inorganic material composition under an electrode potential at a specified pH. This process includes providing an inorganic material composition, having a general formula [AxByCz . . . ]OmHn, where A, B, and C are elements other than O or H, and an electrolyte composition. The electrolyte composition includes salts of A, B, and/or C

As shown in the example of FIG. 2, the Pourbaix diagram 200 may be used to determine the stability of the material. Electrode potential can be adjusted for ensuring stability. Electrode potential and pH of the electrolyte required for stability changes as aqueous A, B, C . . . are added or removed from the electrolyte. A, B, C . . . concentrations in the electrolytes can be utilized to adjust the location of thresholds of stability with respect to electrode potentials and solution pH.

Conditions or parameters of aqueous solution systems within a system are monitored and maintained relative to the inorganic material stability area (or co-precipitation or inorganic material stability area when other metals are processed) with regard to electrochemical (oxidizing) potential (Eh) range and pH range at the prescribed system molarity, temperature, and pressure in order to provide an Eh-pH combination to achieve stable solution equilibrium, as defined by the metal oxide stability area as delineated in, for example, the Pourbaix diagram 200. The process includes selecting an appropriate pH range for use with the particular particular inorganic material, salt, and/or solvent employed by reference to the Pourbaix diagram 200. The lines in the Pourbaix diagram 200 show the equilibrium conditions, that is, where the activities are equal, for the species (or reaction products) on each side of that line. On either side of the line, one form of the species will instead be said to be predominant. The pH, and/or the suitable pH agent, is selected such that the formation of the inorganic material is predominant over the formation of a different species. As shown in FIG. 2, by adjusting A and/or M salt concentration in electrolyte, one can shift or modify the shape of the stability region (as illustrated by arrows shown) for A₂₋ₓMO₃ and hence increase stability.

Based on the Pourbaix diagram, a user, such as a researcher, or an autonomous system may predict a most stable state of an aqueous solution that is in contact with a metal substrate. The stable state may be with respect to electrochemical reaction conditions of solution acidity and an applied electrode bias voltage. In some examples, a Pourbaix diagram may describe the stable form of a compound in a variety of pH and voltage conditions. In some such examples, the Pourbaix diagram may be used to screen thermodynamically stable conditions to predict the presence of corrosion, deposition, or dissolution at a given set of electrochemical conditions. This procedure may require the calculation of reference energies associated with each chemical species to determine their respective chemical potential.

Chemical synthesis systems may use various protocols to identify one or more conditions associated with a chemical and structural similarity that best facilitate deposition of one material onto another. The chemical synthesis system may also be referred to as a physical reactor system. In some examples, a chemical synthesis system uses a topotactic similarity, which describes a match between lattice spacing of surfaces of two atomistic crystal structures. In such examples, the topotactic similarity may be used to estimate the compatibility of two structures for deposition. Additionally, or alternatively, elastic properties associated with a crystal structure may be used to estimate an energetic cost of deforming two lattices into agreement with one another. The estimated energetic cost of deforming may be used to estimate a thermodynamic stability of deposition between one substrate and another. The surface energy associated with different terminations of a substrate may also be used to improve an estimate of a most thermodynamically stable configuration.

Aspects of the present disclosure are directed to combining various protocols to determine appropriate conditions for a desired reaction product. In some aspects, the appropriate conditions for the desired reaction product may be determined based on a search for valid precursors and operating conditions across a range of existing reaction data. Additionally, some aspects may use a recommendation engine that provides a principled suggestion of target reaction engineering strategy. The recommendation engine may consider a broad set of relevant chemical factors. In some examples, the recommendation engine may use information including, but not limited to, a structural similarity of a candidate deposited compound and substrate, an electrochemical stability of a reaction target along many axes of variation, and/or a thermodynamic stability of associated precursors.

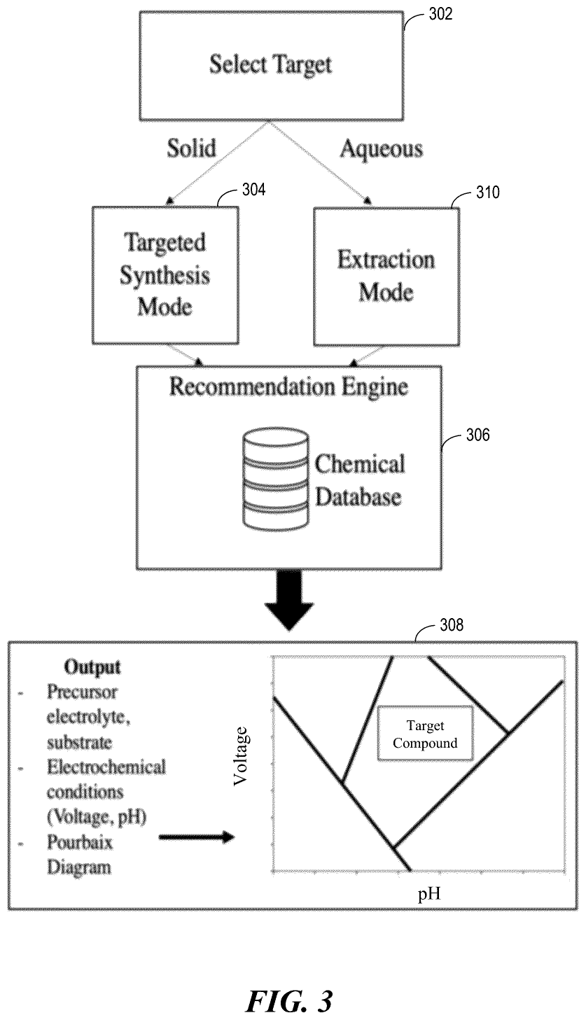

FIG. 3 is a flow diagram illustrating an example of a process 300 for recommending a chemical compound, in accordance with various aspects of the present disclosure. In the example of FIG. 3, the process 300 may be performed by an electrochemical reactor 100 as described with reference to FIG. 1. As shown in FIG. 3, the process 300 may begin at block 302 by selecting a target. The target may be a solid reaction product that is to be synthesized or an aqueous reactant that is to be removed from an aqueous phase. Other types of targets are contemplated by various aspects of the present disclosure. For ease of explanation, the solid reaction product that is to be synthesized may be referred to as a target reaction product. Additionally, the aqueous reactant that is to be removed from the aqueous phase may be referred to as a target aqueous reactant.

As shown in FIG. 3, if the target is the solid reaction product (shown as “solid” in FIG. 3), the process 300 proceeds to a target synthesis mode at block 304. In the target synthesis mode, the process 300 may receive one or more inputs associated with the target reaction product. The one or more inputs may include, but are not limited to, a target crystal structure (e.g., a desired crystal structure), a database of aqueous-ion (e.g., ionic thermochemistry data), solid-state thermochemistry properties (e.g., solid thermochemistry data), and substrate crystal structures currently available for use in the chemical reaction process. The one or more inputs received at block 304 may be provided by a human that is associated with the chemical reaction process. Additionally, the one or more inputs received at block 304 may be processed by a recommendation engine (block 306) that is in communication with a database, such as a chemical database. In some examples, the ionic thermochemistry data and solid thermochemistry data may be stored in the database (e.g., chemical database) prior to receiving the target crystal structure and the substrate crystal structures currently available for use in the chemical reaction process.

In some implementations, for the solid reaction product, the recommendation engine may predict a target reaction condition (e.g., optimal reaction condition) for obtaining the target reaction product. The target reaction condition may be based on a Pourbaix analysis and a structural analysis. The Pourbaix analysis and the structural analysis may be conducted in parallel. In some examples, the Pourbaix analysis may search for aqueous solutions which share common species with the target crystal structure. The Pourbaix analysis may also identify electrochemical conditions associated with the aqueous solutions, where deposition of aqueous solutions (e.g., aqueous ions concentrations) into a target crystal structure may become thermodynamically favored. The structural analysis may identify one or more substrates that may support the target crystal structure. The one or more substrates may be identified based on structural and geometric properties.

At block 308, the recommendation engine generates an output (e.g., recommendation) based on the analysis performed at block 306. In such examples, the recommendation engine may output a substrate crystal structure and composition, and an electrolyte composition. The electrolyte composition may include concentrations of aqueous ionic species, and a voltage and pH that may maximize the kinetic and thermodynamic favorability of a target crystalline compound. The substrate crystal structure and the electrolyte composition provided by the recommendation engine may be used to prescribe an acidity (e.g., pH) and ionic composition (e.g., ions and ion concentrations) of an electrolyte, a particular substrate to host the target crystal structure, and a bias voltage that may be applied to a substrate to make the deposition thermodynamically favorable.

Additionally, as shown in FIG. 3, if the target is the aqueous reactant (shown as “aqueous” in FIG. 3), the process 300 proceeds to an extraction mode at block 310. In the extraction mode, the process 300 receives one or more inputs associated with the target aqueous reactant. The one or more inputs may include, but are not limited to, a target solution species to extract, available host structures (e.g., available target structures), ionic thermochemistry data (e.g., a database of solutions), solid-state thermochemistry data, and substrate structures available to accommodate the host structure. The available substrate structures may be provided to improve the selectivity of a target product. That is, the extraction may change a chemical composition of a host structure, therefore, selectivity of the target product may be improved by providing a stable solid state for the host structure postextraction. As discussed, the ionic thermochemistry data and solid-state thermochemistry data may be received at a database prior to receiving the target solution species to extract, available host structures, and substrate structures available to accommodate the host structure.

The one or more inputs received at block 310 may be provided by a human that is associated with the chemical reaction process. Additionally, the one or more inputs received at block 310 may be processed by a recommendation engine (block 306) that is in communication with a database, such as a chemical database.

In some implementations, for the target aqueous reactant, the recommendation engine may predict optimal extraction conditions for a particular species of interest (e.g., the aqueous reactant targeted for removal). The target aqueous reactant may be based on a Pourbaix analysis and a structural analysis. The Pourbaix analysis and the structural analysis may be conducted in parallel. In some examples, the Pourbaix analysis may search for aqueous solutions which share common species with the target aqueous reactant. The Pourbaix analysis may also identify electrochemical conditions associated with the target aqueous reactant. Furthermore, the Pourbaix analysis may search for one or both of an amount of energy for formation of a precipitate associated with target aqueous reactant or ion species and/or concentrations that support the target aqueous reactant. The structural analysis may identify one or more substrates that may support the target aqueous reactant. The one or more substrates may be identified based on structural and geometric properties.

At block 308, the recommendation engine generates an output (e.g., recommendation output) based on the analysis performed at block 306. In such examples, for the target aqueous reactant, the recommendation engine may output a substrate crystal structure and composition, and an electrolyte composition. The electrolyte composition may include concentrations of aqueous ionic species, and a voltage and pH that may maximize the kinetic and thermodynamic favorability of a target aqueous reactant. The substrate crystal structure and the electrolyte composition provided by the recommendation engine may be used to prescribe an acidity (e.g., pH) and ionic composition (e.g., ions and ion concentrations) of an electrolyte, a particular substrate to host the target aqueous reactant, and a bias voltage that may be applied to a substrate to make the deposition thermodynamically favorable. The recommendation engine may also output an optimal target structure.

For both the target aqueous reactant and the target reaction product, the recommendation engine may also generate a Pourbaix diagram. The Pourbaix diagram and associated target conditions may use results of first-principles calculations, such as a density functional theory, or known experimental and thermochemical data from one or more databases, such as the inorganic crystal structure database (ICSD) or a database associated with the National Institute of Standards and Technology (NIST), to compute the enthalpies of relevant reaction products and intermediates which parametrize a Nernst equation. The compositions with the lowest free energies at a given electrochemical configuration may be considered the most thermodynamically stable. The thermodynamic stability may be used to identify a particular combination of operating condition(s), electrolyte(s), and substrate(s). That is, the Pourbaix diagram may be used to identify a combination of operating condition(s), electrolyte(s), and substrate(s) that yield the highest thermodynamic stability.

The Pourbaix diagram may be generated by extant methods, which may be used to guide the synthesis in a variety of ways. For example, the extant methods may maximize an area of the Pourbaix diagram that stabilizes a target reaction product to accommodate experimental imperfections which result in a deviation from an idealized form. The maximized areas may be areas associated with possible voltage and pH ranges. The voltages, acidity, and ionic concentration conditions provided by the recommendation engine may be used to guide the development of a physical reactor system, such as the system described in FIG. 1.

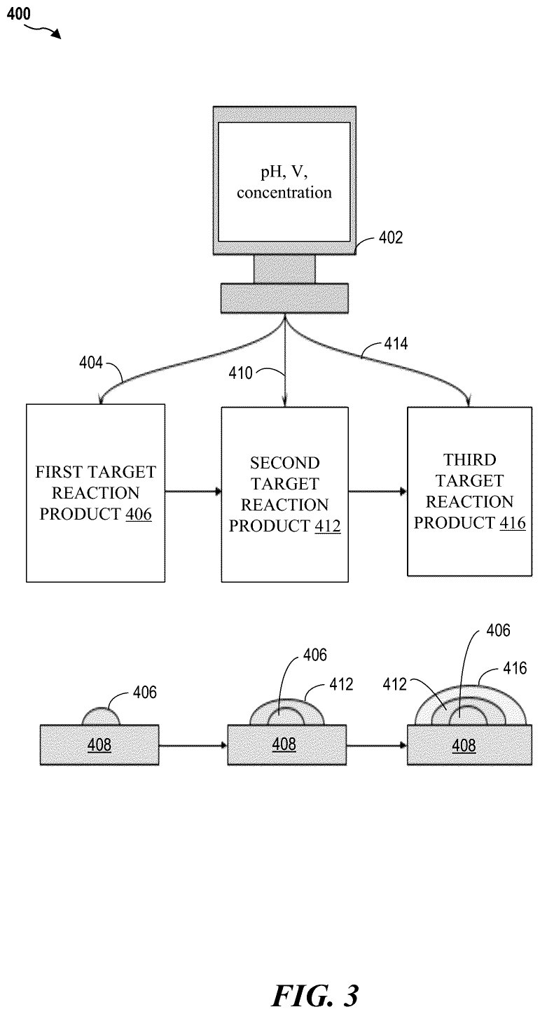

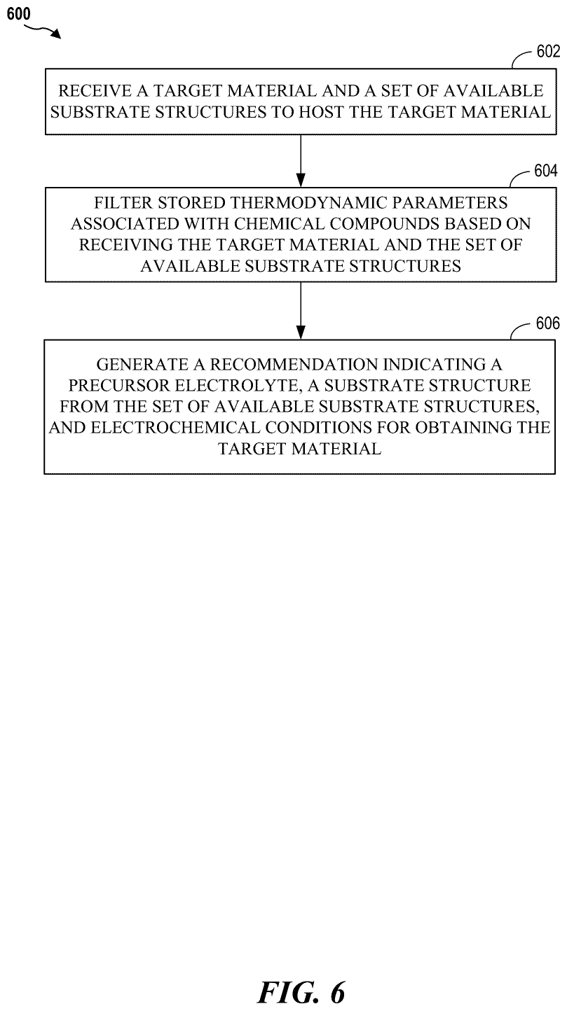

In some implementations, various aspects of the present disclosure may be used to identify and/or optimize reaction protocols for a multi-stage reaction that involves an interaction between an aqueous medium and a substrate. FIG. 4 is a block diagram illustrating an example of a physical reactor system 400, in accordance with aspects of the present disclosure. In the example of FIG. 4, the physical reactor system 400 may use a chemical reaction system 402 specified to guide the synthesis of a solid reaction product. The chemical reaction system 402 is not limited to synthesis of the solid reaction product and may also be used for other tasks, such as guiding the process for removing an aqueous reactant from an aqueous phase. The chemical reaction system 402 may include one or more hardware devices, such as the electrochemical reactor 100 described with reference to FIG. 1, and/or software. One or both of the hardware or software may be non-generic and tailored to perform operations according to aspects of the present disclosure, such as the operations described with reference to the process 300 of FIG. 3 and/or the process 600 of FIG. 6.

As shown in the example of FIG. 4, the chemical reaction system 402 may be used to provision a multi-stage reaction. In some examples, the multi-stage reaction may use an iterative process to form the solid reaction product or remove an aqueous reactant from an aqueous phase. As shown in FIG. 4, at a first stage 404 of the multi-stage reaction, a user may provide one or more inputs associated with a first target reaction product 406. The inputs associated with the first target reaction product 406 may include the inputs described at block 302 of FIG. 3. Based on the inputs provided at the first stage 404, the chemical reaction system 402 may output a substrate crystal structure and composition, an electrolyte composition including concentrations of aqueous ionic species, and a voltage and an acidity (pH) which maximize the kinetic and thermodynamic favorability of a target crystalline compound. The first target reaction product 406 may be formed on a substrate 408 using the combination of operating conditions, electrolytes, and substrate provided by the chemical reaction system 402 at the first stage 404.

In the example of FIG. 4, at a second stage 410 of the multi-stage reaction, the user may provide one or more inputs associated with a second target reaction product 412. The inputs associated with the first target reaction product 406 may include the inputs described at block 304 or 310 of FIG. 3. Additionally, the second target reaction product 412 may be used as an input corresponding to an available substrate structure for the second target reaction product 412. Based on the inputs provided at the first stage 404, the chemical reaction system 402 may output an electrolyte composition including concentrations of aqueous ionic species, and a voltage and an acidity (pH) which maximize the kinetic and thermodynamic favorability of a target crystalline compound. The second target reaction product 412 may be formed on the first target reaction product 406 using the combination of operating conditions and electrolytes provided by the chemical reaction system 402 at the third stage 414.

Finally, as shown in the example 400 of FIG. 4, at a third stage 414 of the multi-stage reaction, the user may provide one or more inputs associated with a third target reaction product 416. The inputs associated with the target reaction product 416 may include the inputs described at block 304 or 310 of FIG. 3. Additionally, the second target reaction product 412 may be used as an input corresponding to an available substrate structure for the third target reaction product 416. Based on the inputs provided at the third stage 414, the chemical reaction system 402 may output an electrolyte composition including concentrations of aqueous ionic species, and a voltage and an acidity (pH) which maximize the kinetic and thermodynamic favorability of a target crystalline compound. The third target reaction product 416 may be formed on second target reaction product 412 using the combination of operating conditions and electrolytes provided by the chemical reaction system 402 at the third stage 414.

The chemical reaction system 402 may selectively query a database of data, such as chemical data, to generate a Pourbaix diagram from a diverse set of chemical species. Furthermore, the chemical reaction system 402 may determine the degree of interfacial similarity between a target solid-state compound and a substrate to recommend one or more conditions, electrolytes, and substrates to use in the physical reactor system 400 to obtain the desired target. More specifically, the chemical reaction system 402 may output a set of optimal conditions of reactions for the synthesis of a solid phase or extraction of a target from an aqueous phase. The set of optimal conditions may include one or more of an electrolyte, a solution pH, applied voltage, and a substrate that are optimal for the rate of synthesis or extraction.

The chemical reaction system 402 may be used to provide computational guidance for various processes, such as, but not limited to, an electrowinning process, an electrowinning-like process, solution purification, or desalination. As discussed, in some examples, the chemical reaction system 402 may be used to design multiple-stage reactor devices, or single-stage reactor devices, which entail a sequence of interactions between electrodes and solution. The reactor devices may use various protocols, such as cycling, transport, or injections to the solution, mechanical re-arrangement of treatment of electrodes, or a variation of electric potential on the electrodes, to achieve a desired purification or synthesis.

In some examples, the chemical reaction system 402 may be used to find a Pareto-optimal electrochemical synthesis for a crystal structure. As an example, the crystal structure may be a chiral antiferromagnet MnSb₂O₆. In this example, the chemical reaction system 402 may recommend a substrate material, a voltage for the chemical reaction, an amount of manganese (Mn) (e.g., ionic concentration), an amount of antimony (Sb) (e.g., ionic concentration), and an acidity to obtain the chiral antiferromagnet MnSb₂O₆.

In this example, the chemical reaction system 402 may determine the optimal conditions for a range of concentrations of manganese and antimony. In some examples, the range may be [1e⁻⁶, 1e⁻⁵, . . . , 10 Molar (M)]. Specifically, for the range of concentrations, the chemical reaction system 402 may generate a Pourbaix diagram, compute reaction energies, compute parasitic reactions, and collect value parasitic reactions and reaction energies. The chemical reaction system 402 may then identify the Pareto reaction associated with a minimum parasitic reaction and a maximum reaction energy. Additionally, the chemical reaction system 402 may compute a minimal coincident interface area (MCIA) associated with one or more respective substrates, and a topotactic similarity for each substrate. In such examples, the chemical reaction system 402 may identify a Pareto substrate associated with a minimum MCIA and a minimum topotactic similarity.

As discussed, various aspects of the present disclosure may be used to identify and/or optimize reaction protocols for a single-stage or multi-stage reaction that involves an interaction between an aqueous medium and a substrate. In some examples, aspects of the present disclosure may be used to find pareto-optimal electrochemical synthesis for a material, such as an acid stable OER or ORR material. In one such example, the material may be manganese antimonate MnSb₂O₆. In such aspects, a chemical reaction system, such as the chemical reaction system 402 described with reference to FIG. 4, may be used to identify an optimal pH level, an optimal concentration of manganese Mn, an optimal concentration of antimony Sb, an optimal voltage at which to operate the electric chemical cell, and an optimal material for a substrate. In such examples, an optimal value may yield the most results while minimizing resource use.

In some examples, for all concentrations of manganese and antimony from [1e⁻⁶, 1e⁻⁵, . . . , 10 Molar (M)], the chemical reaction system may generate a Pourbaix diagram, compute reaction energies (e.g., ionic precursor energy-target energy), compute parasitic reactions, and collect valid parasitic reactions and reaction energies. The pareto reactions may be determined by identifying a minimum parasitic reaction that is associated with a maximum reaction energy. The chemical reaction system may also compute a minimal coincident interface area (MCIA) of a substrate and topotactic similarities for substrates based on available substrate materials. The chemical reaction system may then identify a pareto substrate that satisfies a minimum MCIA and a minimum topotactic similarity.

Table 1 shows an example of reaction energies and the quantity of parasitic reactions generated by the chemical reaction system based on inputs provided for the desired target. The inputs may be similar to inputs described with reference to blocks 304 or 310 of FIG. 3.

| | | Parasitic | Reaction | | |
|---|---|---|---|---|---|
| Voltage | pH | Reactions | Energy (eV) | [Sb] (M) | [Mn] (M) |
| 0.06 | 10.06 | 0 | 0.162 | 0.001 | 0.0001 |
| 0.12 | 12.58 | 1 | 0.208 | 0.0001 | 1 |
| 0.06 | 12.4 | 2 | 0.239 | 10 | 10 |
| 0.42 | 14.56 | 3 | 0.281 | 0.0001 | 10 |
| 0.60 | 11.68 | 4 | 0.343 | 0.0001 | 10 |
| 0.90 | 9.16 | 5 | 0.397 | 0.0001 | 10 |
| 1.20 | 4.48 | 6 | 0.487 | 0.00001 | 10 |
| 1.56 | −1.64 | 7 | 0.637 | 10 | 10 |

The values of TABLE 1 may be obtained by iterating through a database of values and filtering the values based on the inputs associated with the target reaction. The chemical reaction system may iterate through numerous configurations and provide a set of optimal reactions. Based on the values of TABLE 1, the chemical reaction system may identify a minimum parasitic reaction that is associated with a maximum reaction energy. In the example of TABLE 1, the reaction energy of 0.162 is the highest reaction energy associated with the lowest number of parasitic reactions (e.g., zero). As shown in TABLE 1, other reaction energies may increase the number of parasitic reactions. It may be desirable to reduce a number of parasitic reactions while increasing a reaction energy. Therefore, in the current example, based on zero parasitic reactions yielding a maximum reaction energy, the chemical reaction system may also determine that an optimal pH is 10.06, an optimal voltage is 0.06, an optimal manganese concentration is 0.0001, and an optimal antimony concentration is 0.00100.

Additionally, as discussed, the chemical reaction system may also compute an MCIA of a substrate and topotactic similarities for substrates based on available substrate materials. The chemical reaction system may then identify a pareto substrate that satisfies a minimum MCIA and a minimum topotactic similarity. TABLE 2 is an example of values associated with a topotactic similarity and an epitaxial match. The epitaxial match may be based on the MCIA. That is, the epitaxial match estimates suitability of a particular substrate material in terms of whether it needs to be stretched or compressed in view of the targeted structure. The topotactic similarity is based on a structural similarity and estimates whether an interfacial energy is expected to be small relative to a surface energy.

| | Film | Epitaxial | Toptactic |
|---|---|---|---|
| Substrate Material | Orientation | Match | Similarity |
| PbSe | 001 | 0.069340 | 0.8665525 |
| BaTiO3 | 100 | 0.086000 | 0.335749 |
| YAl O3 | 001 | 0.138680 | 0.267926 |
| Ge3(BiO3)4 | 001 | 0.208020 | 0.221253 |
| NdGaO3 | 100 | 0.257999 | 0.157714 |

In the current example, the barium titanate BaTiO3 substrate material yields a minimum epitaxial match and a minimum topotactic similarity in comparison to the other substrate materials. Therefore, the chemical reaction system may suggest barium titanate as the substrate material.

In some examples, the chemical reaction system may be associated with one or more hardware systems that autonomously adjust chemical concentrations and/or a voltage based on the optimal conditions. In one example, based on the values of TABLES 1 and 2, the chemical reaction system may control one or more hardware systems to set a voltage to 0.06, a pH is 10.06, a manganese concentration to 0.0001 M, and an antimony concentration to 0.00100 M. Furthermore, the one or more hardware systems may obtain a barium titanate substrate for the chemical reaction. The one or more hardware systems may operate without human intervention, such that the chemical reaction process is automated.

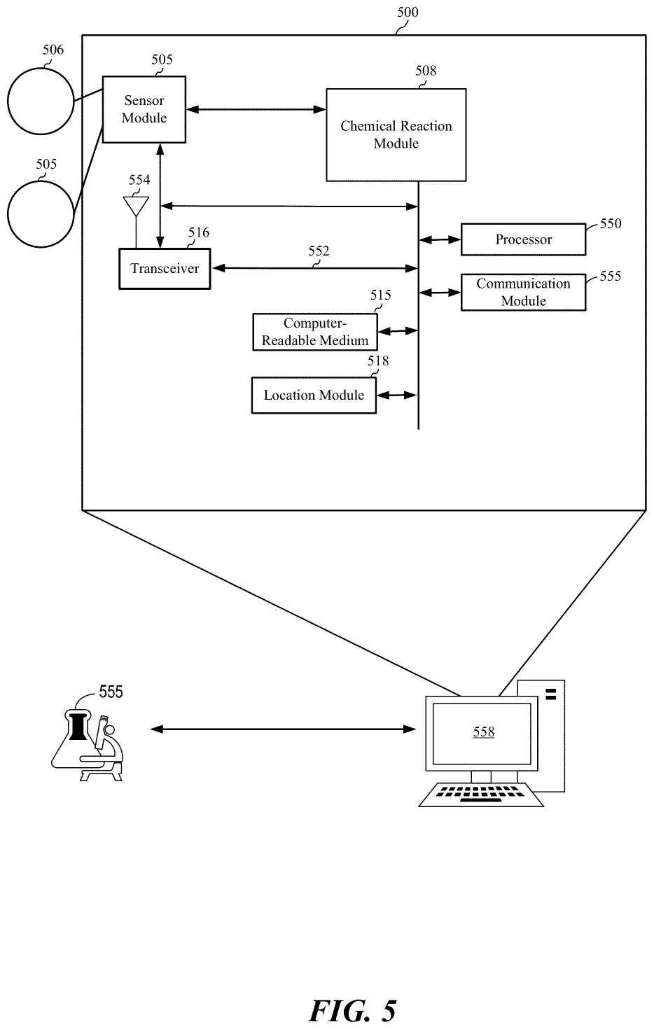

FIG. 5 is a diagram illustrating an example of a hardware implementation for physical reactor system 500 of an active workflow system, according to aspects of the present disclosure. The physical reactor system 500 may also be referred to as a chemical reaction system. The physical reactor system 500 may be a component of a tablet device, user equipment (UE), laptop, desktop, or another type of computing device. For example, as shown in FIG. 5, the physical reactor system 500 is a component of a computing device 558. Aspects of the present disclosure are not limited to the physical reactor system 500 being a component of the computing device 558. Additionally, as described, the active workflow system may be implemented on one or more components within a variety of environments. In some examples, one or more hardware components may be used to implement the active workflow system.

The physical reactor system 500 may be implemented with a bus architecture, represented generally by a bus 552. The bus 552 may include any number of interconnecting buses and bridges depending on the specific application of the physical reactor system 500 and the overall design constraints. The bus 552 links together various circuits including one or more processors and/or hardware modules, represented by a processor 550, a communication module 555, a location module 518, a sensor module 505, an acceleration module 556, and a computer-readable medium 515. The bus 552 may also link various other circuits such as timing sources, peripherals, voltage regulators, and power management circuits, which are well known in the art, and therefore, will not be described any further.

The physical reactor system 500 includes a transceiver 516 coupled to the processor 550, the sensor module 505, a chemical reaction module 508, the communication module 555, the location module 518, the acceleration module 556, and the computer-readable medium 515. The transceiver 516 is coupled to an antenna 554. The transceiver 516 communicates with various other devices over one or more communication networks, such as an infrastructure network, a local area network, a wide area network, a cellular communication network, or another type of network. As an example, the transceiver 516 may transmit received data to a remote storage device for storage.

The physical reactor system 500 includes the processor 550 coupled to the computer-readable medium 515. The processor 550 performs processing, including the execution of software stored on the computer-readable medium 515 providing functionality according to the disclosure. For example, the processor 550, working in conjunction with one or more of the modules 505, 506, 508, 515, 516, 518, 550, 554 555, 556, may execute the software to causes the physical reactor system 500 to perform the various functions described with reference to electrochemical reactor 100 of FIG. 1, the process 300 of FIG. 3, and/or the process 600 of FIG. 6. The computer-readable medium 515 may also store data that is manipulated by the processor 550 when executing the software.

The sensor module 505 may be used to obtain measurements via different sensors, such as a first sensor 506 and a second sensor 505. The first sensor 506 may be a temperature sensor, humidity sensor, and/or another type of environmental sensor. The second sensor 505 may be an environmental sensor or another type of sensor, such as a motion sensor. Of course, aspects of the present disclosure are not limited to the aforementioned sensors as other types of sensors, such as, for example, a camera, a thermal sensor, sonar sensor, and/or lasers are also contemplated for either of the sensors 505, 506.

The measurements of the first sensor 506 and the second sensor 505 may be processed by one or more of the processor 550, the sensor module 505, the chemical reaction module 508, the communication module 555, the location module 518, the acceleration module 556, in conjunction with the computer-readable medium 515 to implement the functionality described herein. In one configuration, the data captured by the first sensor 506 and the second sensor 505 may be transmitted to an external device via the transceiver 516. The first sensor 506 and the second sensor 505 may be coupled to the tablet 558 or may be in communication with the tablet 558.

The location module 518 may be used to determine a location of the tablet 558. For example, the location module 518 may use a global positioning system (GPS) to determine the location of the tablet 558. The communication module 555 may be used to facilitate communications via the transceiver 516. For example, the communication module 555 may be configured to provide communication capabilities via different wireless protocols, such as WiFi, long term evolution (LTE), 2G, IoT, etc. The communication module 555 may also be used to communicate with other components of the tablet 558 that are not modules of the component 200.

The chemical reaction module 508 works in conjunction with the processor 330, the communication module 333, and/or the computer-readable medium 313. The chemical reaction module 508 may be configured to perform operations including operations of the process 600 described below with reference to FIG. 6. In some examples, physical reactor system 500 may work in conjunction with laboratory hardware 590 that facilitates target synthesis or target solution extraction as described in various aspects of the present disclosure. The laboratory hardware 590 may operate in an autonomous mode that does not use a human to perform the target synthesis or target solution extraction. In some examples, the chemical reaction module 508 may work in conjunction with one or more of the modules of the physical reactor system 500 to receives a target material and a set of available substrate structures to host the target material. Additionally, working in conjunction with one or more of the modules of the physical reactor system 500, the chemical reaction module 508 may store thermodynamic parameters associated with chemical compounds based on receiving the target material and the set of available substrate structures. Finally, working in conjunction with one or more of the modules of the physical reactor system 500, the chemical reaction module 508 may generate a recommendation indicating a precursor electrolyte, a substrate structure from the set of available substrate structures, and electrochemical conditions for obtaining the target material, the recommendation may be generated based on filtering the stored thermodynamic parameters.

FIG. 6 illustrates a diagram illustrating an example process 600 performed by chemical reaction hardware, in accordance with aspects of the present disclosure. The chemical reaction hardware may be an example of the laboratory hardware 590 or the physical reactor system 500 described with reference to FIG. 5. The example process 600 is an example of setting parameters for a chemical reaction. As shown in the example of FIG. 6, the process begins at block 602 by receiving a target material and a set of available substrate structures to host the target material. In some examples, the target material is a target crystal structure intended to be synthesized on one of the substrate structures from the set of available substrate structures.

At block 604, the process 600 filters stored thermodynamic parameters associated with chemical compounds based on receiving the target material and the set of available substrate structures. The thermodynamic parameters may include ionic thermochemistry data and solid thermochemistry data. Furthermore, the ionic thermochemistry data may include a group of aqueous ion solutions. Additionally, the solid thermochemistry data may include a group of substrate structures, each substrate structure may be associated with structural and geometric properties. In some examples, filtering the thermodynamic parameters includes filtering the ionic thermochemistry data to identify the precursor electrolyte and the electrochemical conditions; and filtering the precursor electrolyte to identify the substrate structure.

In some examples, filtering the ionic thermochemistry data includes: filtering the group of aqueous ion solutions to identify the aqueous ion solution that shares common species with the target crystal structure; and filtering a group of concentrations of the aqueous ion solution to identify a concentration that satisfies a solution selection condition. Furthermore, filtering the solid thermochemistry data may include: identifying a set of substrate structures from the group of substrate structures; and filtering the set of substrate structures to identify the substrate structure based on the substrate structure satisfying a substrate selection condition. In some examples, each concentration of the group of concentrations may be associated with a voltage, a pH, a parasitic reaction, and a reaction energy. In some such examples, the concentration that satisfies the solution selection condition is associated with a minimum parasitic reaction that returns a maximum reaction energy.

Furthermore, the precursor electrolyte may be associated with the concentration that satisfies the solution selection condition. In some examples, each substrate structure of the set of set of substrate structures may correspond to one available substrate structure of the set of available substrate structures. Additionally, each substrate structure may be associated with a topotactic similarity to the target material and an epitaxial match with the target material.

At block 606, the process 600 generates a recommendation indicating a precursor electrolyte, a substrate structure from the set of available substrate structures, and electrochemical conditions for obtaining the target material, the recommendation may be generated based on filtering the stored thermodynamic parameters.

In some examples, at block 602, the process also receives one or more available target structures. In such examples, the target material may be a target solution to extract from the precursor electrolyte. In some such examples, the precursor electrolyte may be an aqueous solution that is exposed to the solid-state compound with an associated bias voltage. When the target material is a target solution, aspects of the present disclosure induce an interaction between an aqueous electrolyte and a surface compound. After the reaction occurs, the target material (e.g., target solution) may be pulled-out from the now-modified electrolyte solution. Additionally, the target material may be hosted on one of the substrate structures from the set of available substrate structures. Furthermore, the recommendation further recommends a target structure, from the one or more available target structures, that will be in contact with the precursor electrolyte.

Based on the teachings, one skilled in the art should appreciate that the scope of the present disclosure is intended to cover any aspect of the present disclosure, whether implemented independently of or combined with any other aspect of the present disclosure. For example, an apparatus may be implemented or a method may be practiced using any number of the aspects set forth. In addition, the scope of the present disclosure is intended to cover such an apparatus or method practiced using other structure, functionality, or structure and functionality in addition to, or other than the various aspects of the present disclosure set forth. It should be understood that any aspect of the present disclosure may be embodied by one or more elements of a claim.

The word “exemplary” is used herein to mean “serving as an example, instance, or illustration.” Any aspect described herein as “exemplary” is not necessarily to be construed as preferred or advantageous over other aspects.

Although particular aspects are described herein, many variations and permutations of these aspects fall within the scope of the present disclosure. Although some benefits and advantages of the preferred aspects are mentioned, the scope of the present disclosure is not intended to be limited to particular benefits, uses or objectives. Rather, aspects of the present disclosure are intended to be broadly applicable to different technologies, system configurations, networks and protocols, some of which are illustrated by way of example in the figures and in the following description of the preferred aspects. The detailed description and drawings are merely illustrative of the present disclosure rather than limiting, the scope of the present disclosure being defined by the appended claims and equivalents thereof.

As used herein, the term “determining” encompasses a wide variety of actions. For example, “determining” may include calculating, computing, processing, deriving, investigating, looking up (e.g., looking up in a table, a database or another data structure), ascertaining and the like. Additionally, “determining” may include receiving (e.g., receiving information), accessing (e.g., accessing data in a memory) and the like. Furthermore, “determining” may include resolving, selecting, choosing, establishing, and the like.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logical blocks, modules and circuits described in connection with the present disclosure may be implemented or performed with a processor specially configured to perform the functions discussed in the present disclosure. The processor may be a neural network processor, a digital signal processor (DSP), an application specific integrated circuit (ASIC), a field programmable gate array signal (FPGA) or other programmable logic device (PLD), discrete gate or transistor logic, discrete hardware components or any combination thereof designed to perform the functions described herein. Alternatively, the processing system may comprise one or more neuromorphic processors for implementing the neuron models and models of neural systems described herein. The processor may be a microprocessor, controller, microcontroller, or state machine specially configured as described herein. A processor may also be implemented as a combination of computing devices, e.g., a combination of a DSP and a microprocessor, a plurality of microprocessors, one or more microprocessors in conjunction with a DSP core, or such other special configuration, as described herein.

The steps of a method or algorithm described in connection with the present disclosure may be embodied directly in hardware, in a software module executed by a processor, or in a combination of the two. A software module may reside in storage or machine readable medium, including random access memory (RAM), read only memory (ROM), flash memory, erasable programmable read-only memory (EPROM), electrically erasable programmable read-only memory (EEPROM), registers, a hard disk, a removable disk, a CD-ROM or other optical disk storage, magnetic disk storage or other magnetic storage devices, or any other medium that can be used to carry or store desired program code in the form of instructions or data structures and that can be accessed by a computer. A software module may comprise a single instruction, or many instructions, and may be distributed over several different code segments, among different programs, and across multiple storage media. A storage medium may be coupled to a processor such that the processor can read information from, and write information to, the storage medium. In the alternative, the storage medium may be integral to the processor.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

The functions described may be implemented in hardware, software, firmware, or any combination thereof. If implemented in hardware, an example hardware configuration may comprise a processing system in a device. The processing system may be implemented with a bus architecture. The bus may include any number of interconnecting buses and bridges depending on the specific application of the processing system and the overall design constraints. The bus may link together various circuits including a processor, machine-readable media, and a bus interface. The bus interface may be used to connect a network adapter, among other things, to the processing system via the bus. The network adapter may be used to implement signal processing functions. For certain aspects, a user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further.

The processor may be responsible for managing the bus and processing, including the execution of software stored on the machine-readable media. Software shall be construed to mean instructions, data, or any combination thereof, whether referred to as software, firmware, middleware, microcode, hardware description language, or otherwise.

In a hardware implementation, the machine-readable media may be part of the processing system separate from the processor. However, as those skilled in the art will readily appreciate, the machine-readable media, or any portion thereof, may be external to the processing system. By way of example, the machine-readable media may include a transmission line, a carrier wave modulated by data, and/or a computer product separate from the device, all which may be accessed by the processor through the bus interface. Alternatively, or in addition, the machine-readable media, or any portion thereof, may be integrated into the processor, such as the case may be with cache and/or specialized register files. Although the various components discussed may be described as having a specific location, such as a local component, they may also be configured in various ways, such as certain components being configured as part of a distributed computing system.

The machine-readable media may comprise a number of software modules. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a special purpose register file for execution by the processor. When referring to the functionality of a software module below, it will be understood that such functionality is implemented by the processor when executing instructions from that software module. Furthermore, it should be appreciated that aspects of the present disclosure result in improvements to the functioning of the processor, computer, machine, or other system implementing such aspects.

If implemented in software, the functions may be stored or transmitted over as one or more instructions or code on a computer-readable medium. Computer-readable media include both computer storage media and communication media including any storage medium that facilitates transfer of a computer program from one place to another.

Further, it should be appreciated that modules and/or other appropriate means for performing the methods and techniques described herein can be downloaded and/or otherwise obtained by a user terminal and/or base station as applicable. For example, such a device can be coupled to a server to facilitate the transfer of means for performing the methods described herein. Alternatively, various methods described herein can be provided via storage means, such that a user terminal and/or base station can obtain the various methods upon coupling or providing the storage means to the device. Moreover, any other suitable technique for providing the methods and techniques described herein to a device can be utilized.

It is to be understood that the claims are not limited to the precise configuration and components illustrated above. Various modifications, changes, and variations may be made in the arrangement, operation, and details of the methods and apparatus described above without departing from the scope of the claims.

What is claimed is:

1. A method for setting parameters for a chemical reaction, comprising:
receiving a target material and a set of available substrate structures to host the target material;
filtering stored thermodynamic parameters associated with chemical compounds based on receiving the target material and the set of available substrate structures; and
generating a recommendation indicating a precursor electrolyte, a substrate structure from the set of available substrate structures, and electrochemical conditions for obtaining the target material, the recommendation being generated based on filtering the stored thermodynamic parameters.

2. The method of claim 1, further comprising:
generating, via an autonomous device, the precursor electrolyte and the substrate from the material based on the recommendation; and
setting, via the autonomous device, the electrochemical conditions based on the recommendation.

3. The method of claim 1, wherein the target material is a target crystal structure intended to be synthesized on one of the substrate structures from the set of available substrate structures.

4. The method of claim 1, further comprising receiving one or more available target structures,
wherein:
the target material is a target solution to extract from the precursor electrolyte;
the target material is hosted on one of the substrate structures from the set of available substrate structures; and
the recommendation further recommends a target structure, from the one or more available target structures, that will be in contact with the precursor electrolyte.

5. The method of claim 1, wherein:
the thermodynamic parameters comprise ionic thermochemistry data and solid thermochemistry data;
the ionic thermochemistry data comprises a plurality of aqueous ion solutions; and
the solid thermochemistry data comprises a plurality of substrate structures, each substrate structure associated with structural and geometric properties.

6. The method of claim 5, wherein:
filtering the thermodynamic parameters comprises:
filtering the ionic thermochemistry data to identify the precursor electrolyte and the electrochemical conditions; and
filtering the precursor electrolyte to identify the substrate structure.

7. The method of claim 6, wherein:
filtering the ionic thermochemistry data comprises:
filtering the plurality of aqueous ion solutions to identify the aqueous ion solution that shares common species with the target crystal structure; and
filtering a plurality of concentrations of the aqueous ion solution to identify a concentration that satisfies a solution selection condition; and
filtering the solid thermochemistry data comprises:
identifying a set of substrate structures from the plurality of substrate structures; and
filtering the set of substrate structures to identify the substrate structure based on the substrate structure satisfying a substrate selection condition.

8. The method of claim 7, wherein:
each concentration of the plurality of concentrations is associated with a voltage, a pH, a parasitic reaction, and a reaction energy;
the concentration that satisfies the solution selection condition is associated with a minimum parasitic reaction that returns a maximum reaction energy; and
the precursor electrolyte is associated with the concentration that satisfies the solution selection condition.

9. The method of claim 7, wherein:
each substrate structure of the set of set of substrate structures corresponds to one available substrate structure of the set of available substrate structures;
each substrate structure is associated with a topotactic similarity to the target material and an epitaxial match with the target material.

10. An apparatus for setting parameters for a chemical reaction, comprising:
a processor; and
a memory coupled with the processor and storing instructions operable, when executed by the processor, to cause the apparatus to:
receive a target material and a set of available substrate structures to host the target material;
filter stored thermodynamic parameters associated with chemical compounds based on receiving the target material and the set of available substrate structures; and
generate a recommendation indicating a precursor electrolyte, a substrate structure from the set of available substrate structures, and electrochemical conditions for obtaining the target material, the recommendation being generated based on filtering the stored thermodynamic parameters.

11. The apparatus of claim 10, wherein execution of the instructions further cause the apparatus to:
generate, via an autonomous device, the precursor electrolyte and the substrate from the material based on the recommendation; and
set, via the autonomous device, the electrochemical conditions based on the recommendation.

12. The apparatus of claim 10, wherein the target material is a target crystal structure intended to be synthesized on one of the substrate structures from the set of available substrate structures.

13. The apparatus of claim 10, wherein execution of the instructions further cause the apparatus to receive one or more available target structures,
wherein:
the target material is a target solution to extract from the precursor electrolyte;
the target material is hosted on one of the substrate structures from the set of available substrate structures; and
the recommendation further recommends a target structure, from the one or more available target structures, that will be in contact with the precursor electrolyte.

14. The apparatus of claim 10, wherein:
the thermodynamic parameters comprise ionic thermochemistry data and solid thermochemistry data;
the ionic thermochemistry data comprises a plurality of aqueous ion solutions; and
the solid thermochemistry data comprises a plurality of substrate structures, each substrate structure associated with structural and geometric properties.

15. The apparatus of claim 14, wherein execution of the instructions that cause the apparatus to filter the thermodynamic parameters further include instructions to cause the apparatus to:
filter the ionic thermochemistry data to identify the precursor electrolyte and the electrochemical conditions; and
filter the precursor electrolyte to identify the substrate structure.

16. The apparatus of claim 15, wherein execution of the instructions that cause the apparatus to filter the ionic thermochemistry further include instructions to cause the apparatus to:
filter the plurality of aqueous ion solutions to identify the aqueous ion solution that shares common species with the target crystal structure; and
filter a plurality of concentrations of the aqueous ion solution to identify a concentration that satisfies a solution selection condition; and
filter the solid thermochemistry data comprises:
identify a set of substrate structures from the plurality of substrate structures; and
filter the set of substrate structures to identify the substrate structure based on the substrate structure satisfying a substrate selection condition.

17. The apparatus of claim 16, wherein:
each concentration of the plurality of concentrations is associated with a voltage, a pH, a parasitic reaction, and a reaction energy;
the concentration that satisfies the solution selection condition is associated with a minimum parasitic reaction that returns a maximum reaction energy; and
the precursor electrolyte is associated with the concentration that satisfies the solution selection condition.

18. The apparatus of claim 17, wherein:
each substrate structure of the set of set of substrate structures corresponds to one available substrate structure of the set of available substrate structures;
each substrate structure is associated with a topotactic similarity to the target material and an epitaxial match with the target material.

19. A non-transitory computer-readable medium having program code recorded thereon for setting parameters for a chemical reaction, the program code executed by a processor and comprising:
program code to receive a target material and a set of available substrate structures to host the target material;
program code to filter stored thermodynamic parameters associated with chemical compounds based on receiving the target material and the set of available substrate structures; and
program code to generate a recommendation indicating a precursor electrolyte, a substrate structure from the set of available substrate structures, and electrochemical conditions for obtaining the target material, the recommendation being generated based on filtering the stored thermodynamic parameters.

20. The non-transitory computer-readable medium of claim 19, wherein the program code further comprises:
program code to generate, via an autonomous device, the precursor electrolyte and the substrate from the material based on the recommendation; and
program code to set, via the autonomous device, the electrochemical conditions based on the recommendation.
